(19) United States
(12) Patent Application Publication
(10) Pub. No.: US 2015/0351468 A9
(43) Pub. Date: Dec. 10, 2015

(54) Face Mask

(71) Applicant: Bruce Chinquee, Pomona, CA (US)

(72) Inventor: Bruce Chinquee, Pomona, CA (US)

(21) Appl. No.: 14/194,609

(22) Filed: Feb. 28, 2014

Related U.S. Application Data
(60) Provisional application No. 61/771,745, filed on Mar. 1, 2013.

(51) Int. Cl. A41D 13/11; A61F 9/02

(52) U.S. Cl. CPC A41D 13/1184; A61F 9/029

(57) ABSTRACT

A goggle and face-mask assembly includes goggles to which is fastened detachable material including a face protector.

### CROSS-REFERENCE TO RELATED APPLICATIONS

This application claims the benefit of U.S. Provision Application Ser. No. 61/771,745, filed Mar. 1, 2013, the entirety of which is incorporated herein by reference.

### FIELD OF INVENTION

The present invention relates generally to a goggle and face mask assembly, and more specifically to a goggle and face mask assembly that protects the user from harsh weather conditions and/or debris while enabling easy and quick release of the face protection for purposes including oral communication.

### BACKGROUND

Alpine skiing is a popular outdoor sports activity that is normally performed in subfreezing temperatures. During alpine skiing, a skier descends a snow-covered slope of a mountain. Upon reaching the base, the skier can be brought to the mountain peak again through conventional power-operated conveyances. This cycle may be repeated a number of times during a given day. Unfortunately for skiers, the compounding of environmental conditions, like subfreezing temperature and increased weather harshness with increasing altitude and during ascent and descent, compromise the wellbeing and enjoyment of the skier. Those areas which are prone to suffer most from the harsh environmental conditions are those of the skier not shielded by a thermally insulating fabric layer, as, for example, the skier's face.

One way skiers protect their faces from the harsh conditions is by wearing a helmet. While wearing a helmet equipped with a faceplate, the user's eyes are protected from debris and the user's face is protected from the harsh weather. Perhaps most importantly, helmets also protect skiers from head injuries.

Not all skiers opt to wear helmets. When a skier who is wearing a helmet wants to talk to someone else, the skier must remove the faceplate, if possible. If not, he must disengage all the securing mechanisms of the helmet and then remove the helmet, during which time the skier's vision is obstructed. Once the helmet is removed, the user's eyes are again exposed to debris and the harsh weather, both of which are exacerbated by high wind velocity relative to the user when the user is descending the slope at high speeds. Ultimately, this means that a skier who wears a helmet will have trouble speaking with the helmet on, and cannot remove the helmet while descending the slopes because of the visual obstruction during removal and ocular and facial exposure to the harsh weather and debris. Helmets are also costly and relatively difficult to put on and remove.

To more easily communicate, or to save money, some skiers choose to only wear goggles, forgoing the protection and cost of a helmet. However, in doing so, these skiers are protecting only their eyes from the harsh weather and debris. Their face, particularly their nose and mouth regions, is exposed to the harsh elements. This calculus is performed not only by alpine skiers, but anyone who wishes to protect his or her face, like bicyclists and motorcyclists, skateboarders, rollerbladers, and so on.

### SUMMARY OF THE PRESENT INVENTION

The present invention solves the problem of skiers having to choose between significant face protection coupled with difficulty in communication and high purchase price, and ease in communication with low purchase price but minimal face protection.

The present invention protects a user's face whiling enabling the user to easily communicate by allowing the user to quickly and easily reveal his or her mouth. For example, in one embodiment, goggle-mask assembly comprises a goggle, face-mask comprising a kerchief and support structure, and a plurality of magnets to attach the face-mask to the goggles. In this embodiment, the user of the goggle-mask assembly has his or her mouth covered by the kerchief with the kerchief attached to the goggle. The user can also, using for example his or her hands, remove the kerchief partially or completely in order to reveal his or her mouth, for example to speak clearly to a companion. Once the user wishes to cover his or her mouth again, for example to protect his or her mouth from the harsh environment, magnetic means for attaching the kerchief to the goggle allow the user to re-cover his or her mouth without additional tools, and do so quickly and easily.

### BRIEF DESCRIPTION OF THE DRAWINGS

The abovementioned and other features of the invention disclosed herein are described with a reference to the drawings of preferred embodiments. The illustrated embodiments are intended to demonstrate, but not limit the inventions. The drawings contain the following figures:

FIG. 1 is a front view of the goggle and mask components of the present invention.

FIG. 2 is a view of the mask of FIG. 1 in the process of being assembled.

FIG. 2 is a view of the mask of FIG. 1 in a later stage of assembly.

FIG. 4 is a view of the mask of FIG. 1 assembled on the user.

FIG. 5 is a sectional view of the mask of FIG. 4, taken along line 5-5 in the direction of the appended arrows.

FIG. 6 is a front view of a windowed mask and its cover.

FIG. 7 is a view of the mask of FIG. 7 with the window covered.

FIG. 8 is a sectional view of the mask of FIG. 7, taken along line 8-8 in the direction of the appended arrows.

FIG. 9 is a view of the mask of FIG. 7 on the user, window uncovered.

FIG. 10 is a front view of the goggle and mask components of the present inventions. This mask has hooks on each end.

FIG. 11 is a view of the mask of FIG. 10 assembled on the user.

FIG. 12 is a front view of the goggle and mask components from FIG. 10.

### DETAILED DESCRIPTION OF THE PREFERRED EMBODIMENTS

While the present description sets forth specific details of various embodiments, it will be appreciated that the description is illustrative only and should not be construed in any way as limiting.

### Eye, Headwear: Goggles

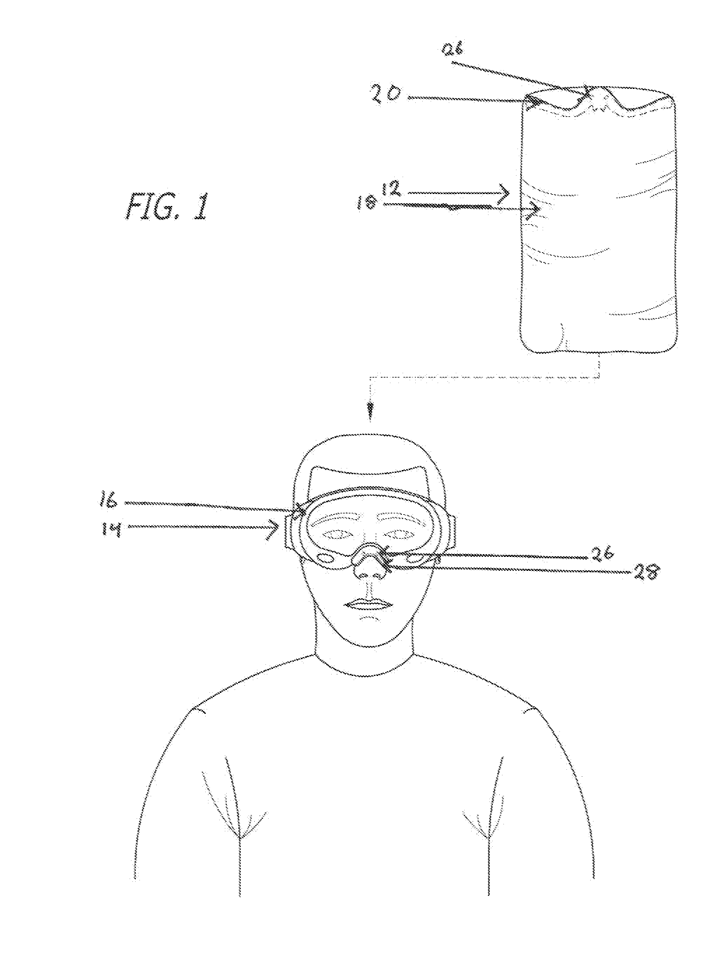

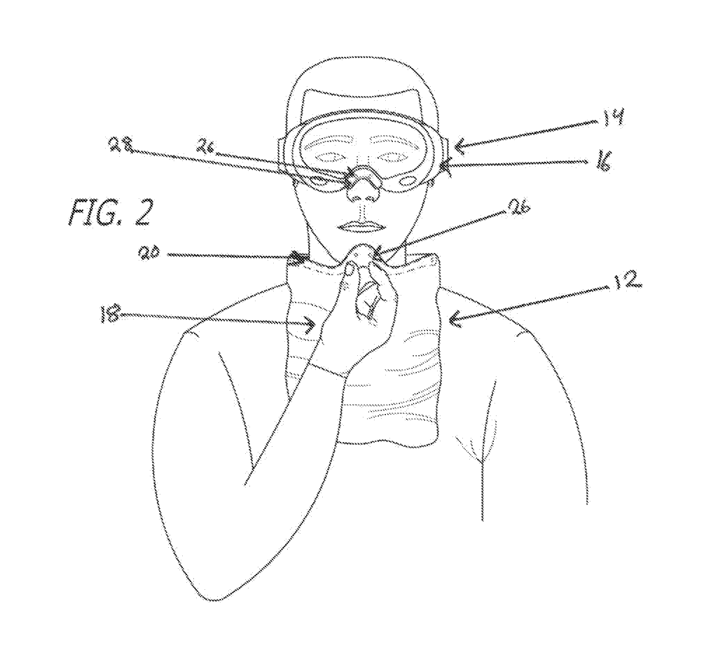

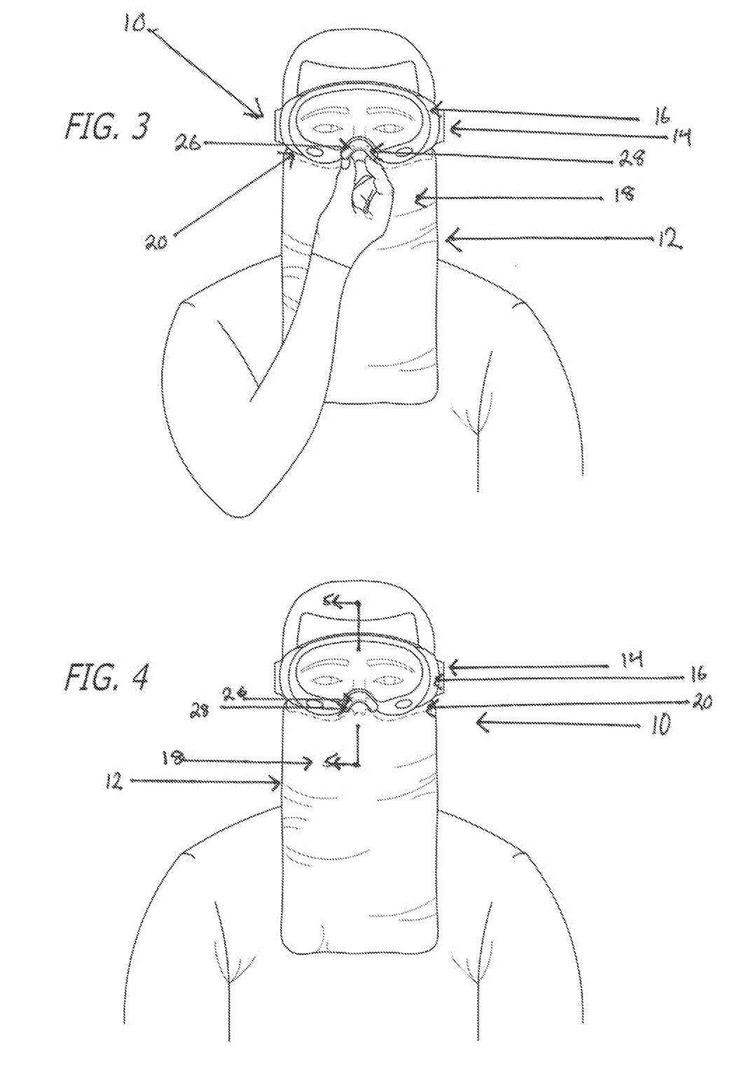

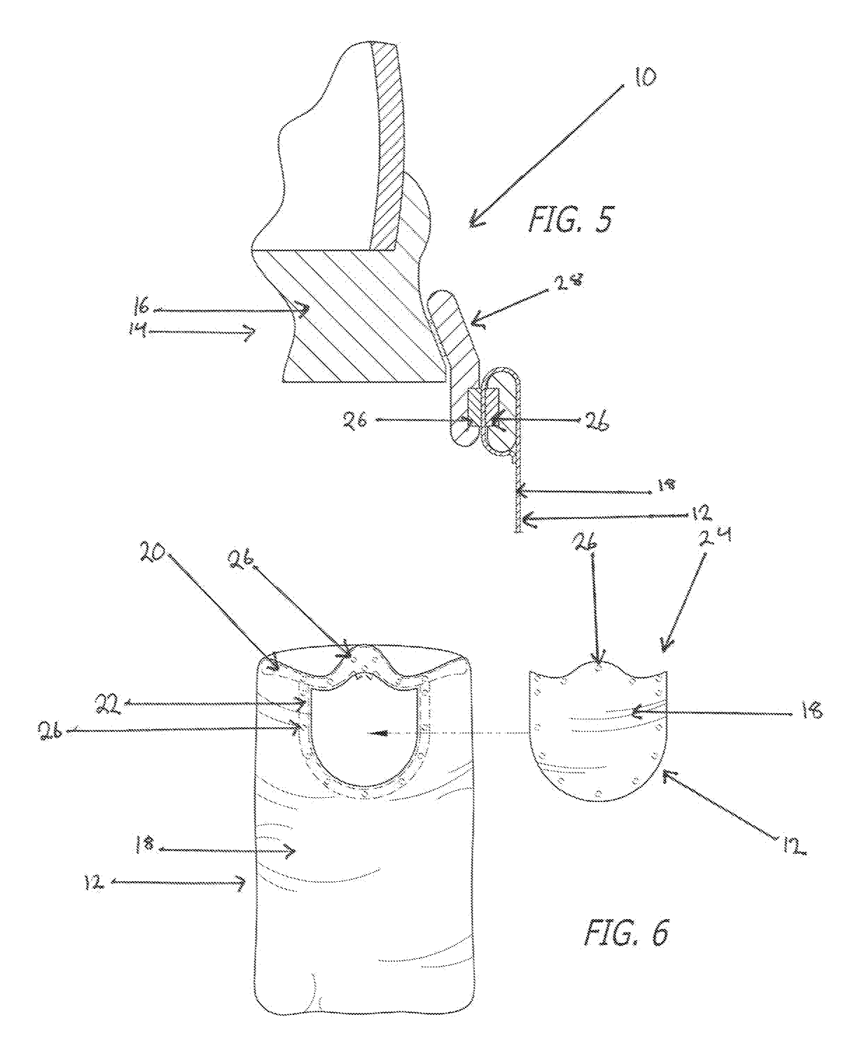

FIGS. 1-5 illustrate a preferred embodiment, the goggle-mask assembly 10 with a tubular fabric face-mask 12. This preferred embodiment incorporates eyewear, such as goggles 14, sunglasses, and prescriptions. The eyewear can be any adornment worn above the bottom of the user's lips. Well-known by those with knowledge in the art, goggles 14 and other eyewear can be constructed a number of ways while still providing its purpose. For example, and without limiting the embodiments, the frame 16 may be metal or may be plastic. The frame 16 may even include, among other elements, a rubber component. The goggle 14 must merely serve the function of protecting a portion of the user's face or head while not completely obstructing the view of the user.

### The Face-Mask

The present invention is designed to have a connection, immediate or otherwise, between goggles 14 and a face-mask 12 where the face-mask 12 protects the mouth and/or nose region of the user while the face-mask 12 can be manipulated to expose user's mouth and/or nose region. Well-known by those with knowledge in the art, the face-mask 12 can be constructed a number of ways while still providing its purpose of protecting the mouth and/or nose region of the user. For example, and without limiting the embodiments, the face-mask 12 can be manufactured from either hard or soft protective material 18. Examples of hard protective material 18 include metal or plastic, or even tempered glass. Examples of soft protective material 18 include fabric, cloth, textiles or any other flexible material. For example, the present invention may be embodied by using a kerchief as the protective material 18 as part of the face-mask 12.

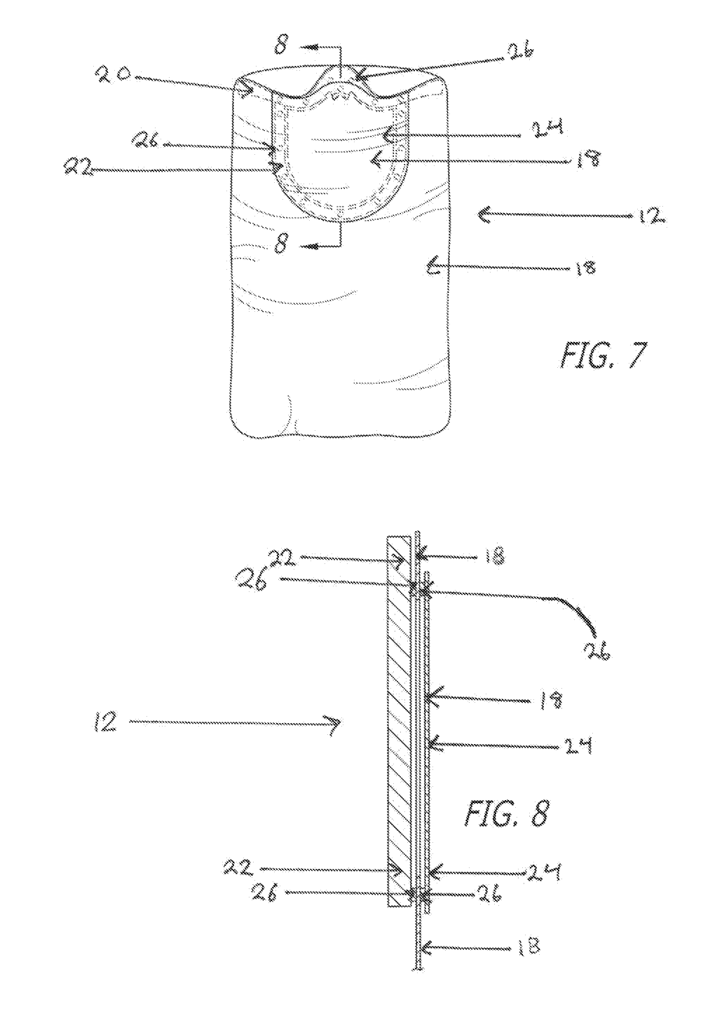

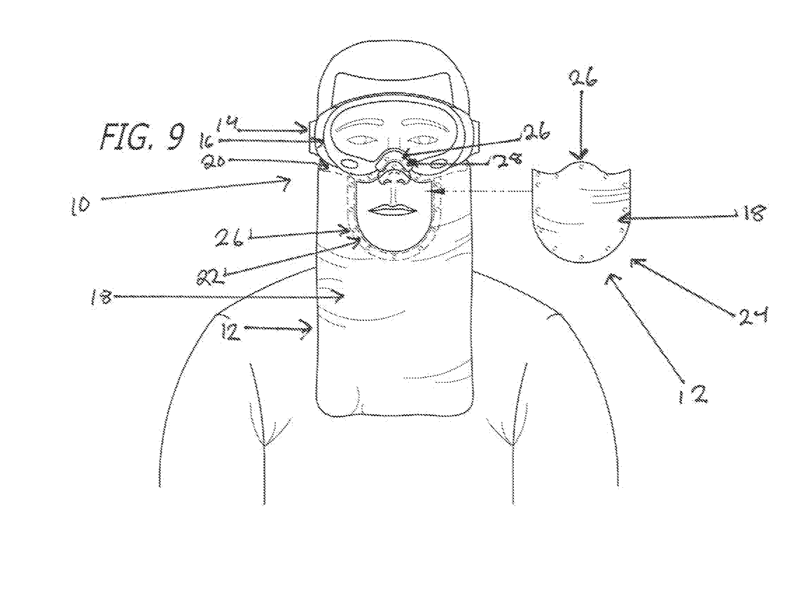

The protective material 18 can be implemented to prevent the protective material 18 from falling off the user. For example, the protective material 18 may be a fabric sleeve, which is vertically tubular. This example is shown in all drawings, FIGS. 1-11. This sleeve can slide over the user's head and onto the user's neck not unlike putting on a T-shirt. This kind of embodiment prevents the face-mask 12 from falling off the user. Another embodiment that achieves this purpose is one where the protective material 18 is restricted from falling off the user by being fastened behind the user's head or neck, by any means of fastening. The face-mask 12 may consist of the protective material 18. However, face-masks may comprise protective material 18 that is supported by a securing frame 20 (a component that is distinct from the goggle frame 16). This securing frame 20 can provide structure to the protective material 18 and/or protect the user's face. The securing frame 20 also provides a foundation for whatever means of attachment is used to connect the face-mask 12 to the goggles 14 (or whatever eye or headpiece is used in the embodiment). Although illustrated in the drawings, the securing frame 20 is not a requirement in an embodiment of the present invention.

### The Face-Mask: Windowed

Another preferred embodiment is illustrated in FIGS. 6-9 The face-mask 12 may also comprise a window within the protective material 18. In this kind of embodiment, the face-mask 12 may have a securing frame 20 that also provides a window frame 22 (also distinct from the goggle frame 16). The window frame 22 enables a window in the protective material 18 that exposes the user to the outside environment. A window cover 24 complements the window frame 22. The window cover 24 detachably connects to the window-frame 22, at which time the user is less exposed to the outside environment. Any means of attachment can be used to detachably connect the window cover 24 to the window frame 22. For example, the window frame 22 and the window cover 24 may be lined with magnets 26 in a way that enables detachable connection between the two.

### Goggle/Face-Mask Attachment: Magnets

One way in which the face-mask 12 can be detachably connected to the goggles 14 is by using magnets 26. In FIG. 1, the goggles 14 has fixed upon it a nose piece 28. In this embodiment, two magnets 26 are attached to a nose-piece 28 that is fastened to the goggles 14 near where the goggles 14 rests upon the user's nose. Although here there are two magnets 26, and they attached to the nose-piece 28, which in turn is attached the bottom of the goggles 14, the magnets 26 need not be in a set of two nor affixed on a nose piece 28. The magnets 26 can be affixed anywhere on the goggles 14 including, but not limited to, anywhere on the goggle frame 16, anywhere on the lens, anywhere on the securing strap of the goggle 14, or any combination thereof. The goggles 14, or any piece or attachment thereof, can also be magnetized instead of attaching magnets 26 to the goggles 14. The magnets 26 can even be affixed to headwear, like a cap, a beanie, ear muffs, or headphones (not shown).

### Goggle/Face-Mask Attachment: Hooks

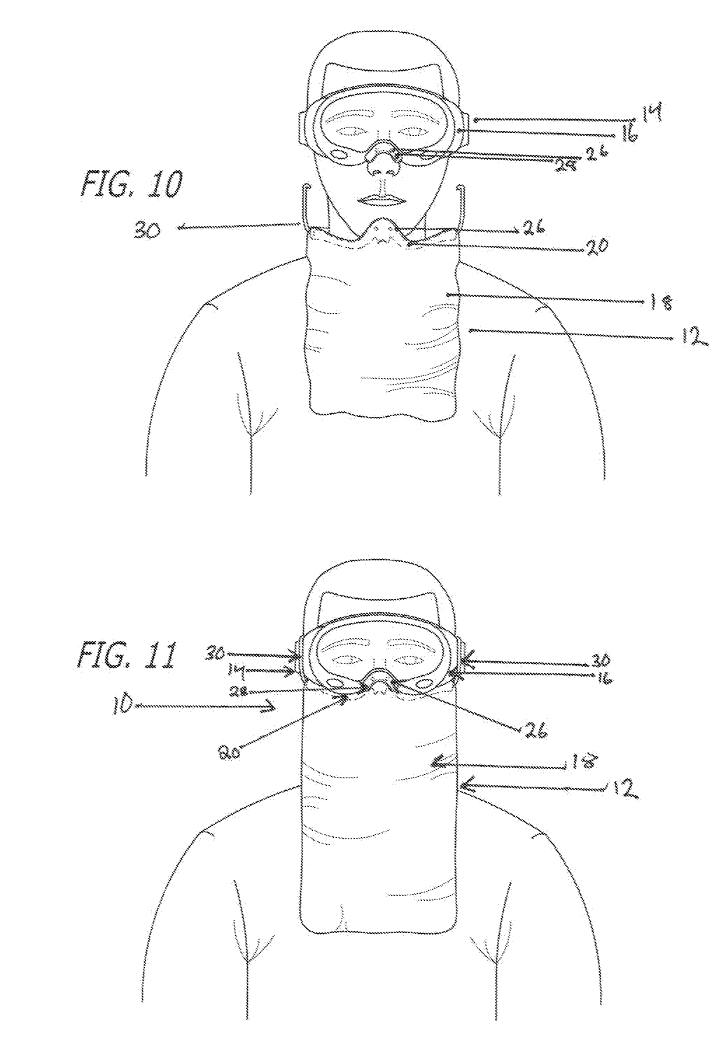

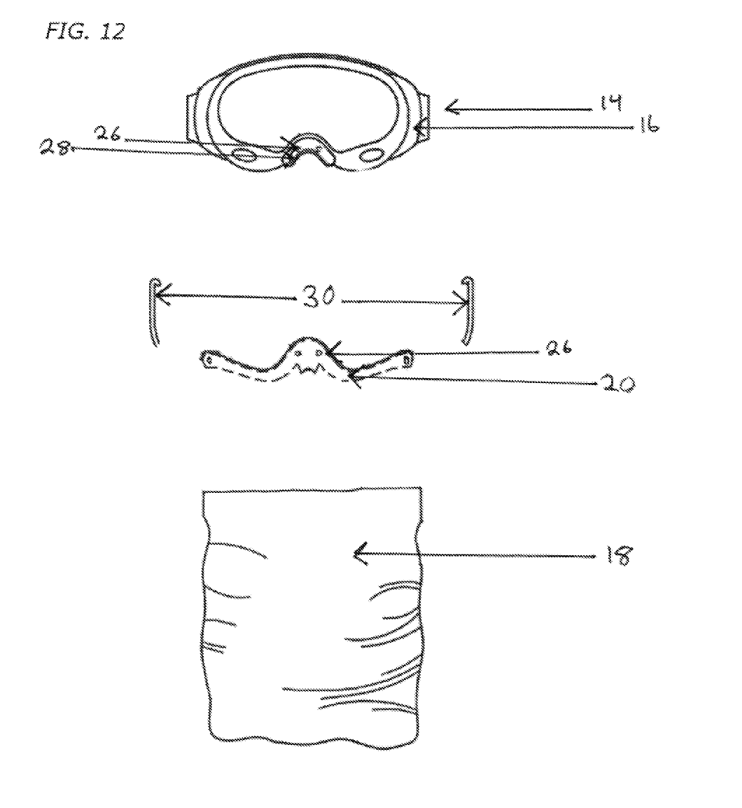

In FIGS. 10-12, the face-mask 12 has hooks 30 attached to its securing frame 20. These hooks 30 serve to supplement or supplant the magnetic 26 attachment mechanism described above. The hooks 30 grab onto the goggles 14. The hooks 30 need not grab onto goggles 14, but any adornment that would serve to supplement or supplant the magnetic 26 attachment mechanism described above. The hooks 30 themselves can be any means of grabbing onto an adornment.

### Goggle/Face-Mask Attachment: Combination and Others

The purpose of the magnets 26 is function as a means to fasten the face-mask 12 to the goggles 14 an embodiment of the present invention. Although in FIG. 1, the means for fastening the face-mask 12 to the nose piece 30 is four magnets 26 (two magnets 26 on the nose piece 28 and two magnets 26 comprised in the face-mask 12), any means for fastening, now invented or invented in the future, can be used to fasten the face-mask 12 to the goggles 14. For example, the means for fastening can be: one or more hook-and-loop fasteners; one or more snap fasteners; one or more zippers; one or more button and shank assemblies; one or more means of clamping, friction-fitting; and; semi-permanent adhesive (not shown here). Moreover, any means for fastening can be used to affix the means for fastening the goggles 14 to the face-mask 12. For example, adhesive (not shown) can be used to affix magnets 26 to the goggles 14, or adhesive (not shown) could be used to affix magnets 26 onto a nose piece 30, which is in turn affixed via adhesive (not shown) onto the goggles 14.

More than one means for fastening the goggles 14 to the face-mask 12 can be used in a single embodiment of the invention. For example, a magnet 26 may be used in conjunction with a zipper (not shown) to fasten the face-mask 12 to the goggles 14. In using this illustrative embodiment, the user can tear away only the portion of the face-mask 12 fastened by the magnet 26 while resting assured that the face-mask 12 easily re-attachable to the goggles 14, in part because the magnet 26 allows for easy reconnection, but also because the portion of the face-mask 12 affixed by the zipper makes sure that the face-mask 12 does not fall away from the user while ensuring that the portion of the face-mask 12 with the magnet 26 is still positioned to line up with the magnet 26 on the goggles 14.

As implied in the illustrative embodiment above, the face-mask 12 does not need to be completely detachable from the goggles 14. For example, a portion of the face-mask 12 may be permanently attached to the goggles 14 while the remainder of the face-mask 12 hangs loosely unless affixed by a means for fastening that allows the user the easily connect the face-mask 12 to the goggles 14. For example, in one embodiment the face-mask 12 may be permanently affixed to the goggles 14 near its securing strap connection on one side while a magnet 26 is affixed to the bottom of the goggles 14 near the securing strap connection on the other side. In this illustrative embodiment, the user can detach a portion of the face-mask 12 thereby revealing the user's mouth, for example to speak more easily. Once a part of the face-mask 12 is detached, the user would not have not hold onto the face-mask 12 but instead the face-mask 12 would remain attached to the goggles 6. Once the user decides to reattach the face-mask 12 to the goggles 6 in full, the user can easily do so by positioning the portion of the face-mask 12 with a magnet 26 near the portion of the goggles 14 with a magnet 26. Moreover, one or more means of fastening can be used to keep the face-mask 12 accessible to the user while the face-mask 12 is not configured to cover the user's mouth.

The protective material 18 is affixed, permanently or detachably, to the face-mask 12, and the face-mask 12 connects, permanently or detachably, to the eyewear (e.g. goggles 14). An embodiment may have the face-mask 12 be movable to facilitate communication, or a different embodiment may have only the protective material 18 be movable with respect to the rest of the facemask 12. Any means of fastening can be used, including but not limited to magnets 26, hook and loop fasteners, clamps, button and shank assemblies, adhesive, friction fit, or any combination of these or any means for fastening. The eyewear frame 16 may be also magnetized to serve as a part of or the whole a means for fastening.

As indicated above, one purpose of the present invention, among other purposes inherent in its design, is to enable a user to quickly and easily reveal and cover his or her mouth. This, among others, is a benefit of the present invention.

What I claim is:

1. A goggle and mask assembly comprising:
goggles, comprising a frame, a lens mounted on said frame, and a means for securing said goggles on a user, wherein said frame is at least large enough to cover the eyes of said user;
detachable material comprising a face protector
means for fastening said detachable material to said goggles;
wherein said means for fastening allows engagement and disengagement in whole or in part of said material with said goggles.

2. A goggle and mask assembly as in claim 1, wherein said material is fabric.

3. A goggle and mask assembly as in claim 1, wherein a portion of said material is permanently affixed to said goggles.

4. A goggle and mask assembly as in claim 1, wherein said goggles further comprises a part of said means for fastening and wherein said material further comprises a part of said means for fastening.

5. A goggle and mask assembly as in claim 1, further comprising:
an attachment arm comprising a means for fastening said attachment arm to said goggles,
a means for fastening said attachment arm to a mask-frame;
said mask-frame comprising a means for fastening in whole or in part of said mask-frame to said detachable material.

6. A goggle and mask assembly as in claim 2, wherein said fabric comprises a generally tubular fabric sleeve having a first open end and a second open end, where the size of one or both said open ends is adjustable to accommodate different size heads and necks of goggle users.

7. A goggle and mask assembly comprising:
goggles, comprising a frame, a lens mounted on said frame, and a means for securing goggles on a user, wherein said frame is at least large enough to cover the eyes of said goggles user;
fabric material;
mask frame attachment;
a first means for fastening said mask frame attachment to said goggles;
a second means for fastening said fabric material mask frame attachment
wherein said first means for fastening allows engagement and disengagement in whole or in part of said material with said mask frame attachment.

8. A goggle and mask assembly as in claim 7, wherein said first means for fastening is one or more magnets and wherein said goggle further comprises one or more magnets.

9. A goggle and mask assembly as in claim 7, further comprising a second attachment, wherein said second attachment is attached by a means for attachment to said goggles and said second attachment is attached by a means for attachment to said mask frame attachment.

10. A goggle and mask assembly as in claim 9, wherein said second means for fastening is one or more magnets and wherein said fabric material comprises one or more magnets.

11. A goggle and mask assembly as in claim 7, wherein the means for fastening, said first means for fastening and said second means for fastening, is one or more hook-and-loop fastener, one or more snap fastener, one or more zipper, one or more button and shank assembly, one or more means of clamping, one or more magnets, means for permanent fixture, semi-permanent adhesive, or friction fit, both first and second means for fastening being the same or being any combination thereof.

12. A goggle and mask assembly as in claim 7, wherein said fabric comprises a generally tubular fabric sleeve having a first open end and a second open end, where the size of one or both said open ends is adjustable to accommodate different size heads and necks of goggle users.

13. A goggle and mask assembly as in claim 9, wherein said fabric comprises a generally tubular fabric sleeve having a first open end and a second open end, where the size of one or both said open ends is adjustable to accommodate different size heads and necks of goggle users.

14. A goggle and mask assembly as in claim 11, wherein said fabric comprises a generally tubular fabric sleeve having a first open end and a second open end, where the size of one or both said open ends is adjustable to accommodate different size heads and necks of goggle users.

15. A goggle and mask assembly comprising:
goggles, comprising a frame, a lens mounted on said frame, and a means for securing goggles on a user, wherein said frame is at least large enough to cover the eyes of said goggles user;
face-mask material,
mask frame attachment;
intermediary attachment;
at least one magnet serving as a means for fastening said mask frame attachment to said goggles;
at least one magnet serving as a means for fastening said material to said goggles;
a means for fastening said fabric material to said mask frame attachment
a means for fastening said intermediary attachment to said goggles.

16. A goggle and mask assembly as in claim 15, wherein said means for fastening said face-mask material to said mask frame attachment comprises adhesive

17. A goggle and mask assembly as in claim 15, wherein said means for fastening said face-mask material to said mask frame attachment comprises use of sewing

18. A goggle and mask assembly as in claim 15, wherein said means for fastening said face-mask material to said mask frame attachment comprises friction, where the face-mask material is held in place against the mask frame.

19. A goggle and mask assembly as in claim 15, wherein said means for fastening said intermediary attachment to said goggles comprises one or more magnets.

20. A goggle and mask assembly as in claim 15, wherein said means for fastening said intermediary attachment to said goggles comprises adhesive.
